(19) United States
(12) Patent Application Publication
(10) Pub. No.: US 2025/0255814 A1
(43) Pub. Date: Aug. 14, 2025

(54) LIPOSOMAL DRUG CARRIER BOUND TO THE BLOOD CELL MEMBRANES, AND PREPARATION METHODS THEREFOR, AND USES THEREOF

(71) Applicant: INSTITUTE OF BLOOD TRANSFUSION, CHINESE ACADEMY OF MEDICAL SCIENCES, Chengdu, Sichuan (CN)

(72) Inventors: Ye CAO, Chengdu, Sichuan (CN); Kehui ZHU, Chengdu, Sichuan (CN); Yingcan XU, Chengdu, Sichuan (CN); Hong WANG, Chengdu, Sichuan (CN); Rui ZHONG, Chengdu, Sichuan (CN); Jiaxin LIU, Chengdu, Sichuan (CN)

(21) Appl. No.: 18/847,717

(22) PCT Filed: Mar. 10, 2023
(86) PCT No.: PCT/CN2023/080763

§ 371 Date: Oct. 23, 2024

(30) Foreign Application Priority Data
Mar. 17, 2022 (CN) 202210277320.6

(51) Int. Cl. A61K 9/1272; A61K 9/1277; A61K 31/337; A61K 31/573; A61K 47/46

(52) U.S. Cl. CPC A61K 9/1272; A61K 9/1277; A61K 31/337; A61K 31/573; A61K 47/46

(57) ABSTRACT

Liposomal drug carriers are obtained by mixing liposomes with blood cells. The liposomes adhere to the surface of the blood cell membranes or fuse with the blood cell membranes. The liposomal drug carriers carry different types of medicaments, with high entrapment efficiency and drug-loading capacity, effectively improving drug delivery efficiency. Moreover, the drug carriers allow liposomes to bind to the cell membranes of bioactive blood cells, and after binding, the blood cells can still maintain their intact morphology as well as good activity and function, that can effectively prolong the half-life of a medicament, better avoid the clearance by the autoimmune system, and achieve better drug release and clinical treatment at lower doses.

### FIELD OF THE INVENTION

The present invention belongs to the technical field of biomaterials, and specifically relates to a liposomal drug carrier bound to the blood cell membranes, as well as preparation methods therefor and uses thereof.

### BACKGROUND OF THE INVENTION

Due to the non-targeted release of broad-spectrum hydrophobic chemotherapeutic drugs, conventional chemotherapy using such drugs often causes serious toxic side effects (hair loss, gastrointestinal reactions, cardiac toxicity, and neurological damage), and lacks tumor-targeting, resulting in unsatisfactory treatment outcomes. In recent years, new particle delivery systems such as drug-loaded nanoparticles, liposomes, and microspheres have received increasing attention due to their obvious advantages of high loading efficiency and controllable release performance. However, because of their short half-lives, high required dosages, and serving as exogenous substances, they are easily cleared by the human immune system after administration, resulting in low bioavailability. A large number of blood cells exist in the blood, and are distributed throughout the body as the flow of blood. Using human blood cells as a new type of drug delivery carrier is expected to effectively avoid the clearance function of the body's immune system, improve bioavailability, and increase half-life to achieve good therapeutic effects at low concentrations.

Red blood cells (RBCs), as endogenous blood cells, can enhance immune escape ability and have the advantages of high bioavailability, good biocompatibility, and long cycle life (45 days and 120 days in mice and humans, respectively). Meanwhile, for RBCs, the unique shape and geometry (high ratio of surface area to volume), as well as the membrane structure and compositions, allow them to have extremely strong deformability and durability. In addition, RBCs are the most abundant blood cells in the blood and are easy to obtain. Therefore, RBCs are a new drug delivery system with high attractiveness and application prospect.

At present, the methods of drug delivery based on blood cells include: directly entrapping drugs or drug-loaded carriers in blood cells by electroporation, hypotonicity, endocytosis, and other methods; secondly, the drug-loaded carriers are bound to the surface of the blood cell membrane or fuse with the blood cell membranes by coupling and other methods. For example, in 1981, Denmark team and Pedro Cabrales team transported doxorubicin (DOX) across membranes into cells by incubating DOX solution with RBCs and electroporation. However, there are some limitations for the available methods: when drugs are directly entrapped in cells, corresponding defects are found in the preparation and delivery processes. For example, several studies have indicated that the biological toxicity of drugs themselves causes real damage to various healthy tissues and organs of the body, which may also cause great damage to blood cells, leading to loss of function. Moreover, for hydrophobic drugs, due to their lipophilic characteristics, the solubility will be greatly reduced, and the therapeutic effect will be significantly weakened; by coupling or electroporation, the drug-loaded carrier can easily change the original membrane structures of blood cells, making it difficult to ensure the release efficiency of the drug. In addition, that can cause hemolysis of RBCs, leading to a decrease in delivery efficiency as well as damage to the integrity of cell structure and function. The drug-loaded carrier is also easily cleared by the immune system in the body and cannot achieve long-term circular delivery. The literature (A splenic-targeted versatile antigen courier: iPSC wrapped in coalescent erythrocyte-liposome as tumor nanovaccine) discloses the erythrocyte membrane is used to fuse with mannose-liposomes. Patent JP5571706B2 discloses the fusion of liposomes and RBCs to transport their contents into cells. However, currently, the basic method of fusing with RBCs is to first extract the erythrocyte ghost, and then the drug is loaded, that can make the RBCs lose their activities, and is unable to have a long circulation time. The above-mentioned problems limit the clinical application of erythrocyte-mediated drug delivery. For blood cell-based drug delivery systems, it is of great significance to study how to use live blood cells to deliver drugs without losing activities and functions of blood cells, and improve the availability of drugs.

### CONTENT OF THE INVENTION

The object of the present invention is to provide a liposomal drug carrier bound to the blood cell membranes, as well as preparation methods therefor and uses thereof.

The present invention provides a liposomal drug carrier bound to the blood cell membranes, which is obtained after mixing liposomes with blood cells; the liposomes adhere to the surface of the blood cell membranes or fuse with the blood cell membranes;

Further, the liposomal drug carriers are obtained by incubating liposomes with blood cells together;
preferably, the incubation time is 1-6 h; and/or, the incubation temperature is 2-40° C.;
more preferably, the incubation time is 4 h; and/or, the incubation temperature is 37° C.

Further, the blood cells are erythrocytes or platelets;
preferably, the blood cells are erythrocytes.

Further, when the liposomes are incubated with blood cells, the liposome concentration is 1-6 μmol, and the number of blood cells is (1-6)×10⁸.
preferably, when the liposomes are incubated with blood cells, the liposome concentration is 2 mol, and the number of blood cells is 3×10⁸.

Further, the liposomes are obtained by mixing one, two or more raw materials selected from egg yolk lecithin, 1-palmitoyl-2-oleoyllecithin, soybean phospholipid, dipalmitoylphosphatidylcholine (DPPC), dioleoylphosphatidylcholine, dioleoylphosphatidylethanolamine (DOPE), dioleoylphosphatidylcholine, and distearoylphosphatidylcholine with cholesterol, and then prepared by thin-film rehydration method;
preferably, the liposomes are prepared from the following raw materials at the indicated weight ratio: 50-60 parts of DPPC and 1-5 parts of cholesterol;
alternatively, the liposomes are prepared from the following raw materials at the indicated weight ratio: 40-50 parts of DPPC, 10-20 parts of DOPE, and 1-5 parts of cholesterol;
more preferably, the liposomes are prepared from the following raw materials at the indicated weight ratio: 59.5 parts of DPPC and 3.5 parts of cholesterol;
alternatively, the liposomes are prepared from the following raw materials at the indicated weight ratio: 46.2 parts of DPPC, 13.4 parts of DOPE, and 3.5 parts of cholesterol.

Further, the method for preparing the liposomes comprises the following steps:
(1) The raw materials for preparing liposomes are dissolved in an organic solvent according to the weight ratio, and then the thin film is obtained by vacuum rotary evaporation using a thin-film method;
(2) The thin film prepared in step (1) is hydrated to obtain the liposomes.

Preferably,
in step (1), the organic solvent is selected from the group consisting of methanol, trichloromethane, ethanol or isopropanol;
and/or, in step (1), the temperature for vacuum rotary evaporation used in the thin-film method is 30-70° C.;
and/or, in step (1), the time for vacuum rotary evaporation used in the thin-film method is 10-60 min;
and/or, in step (2), the solvent used for hydration is PBS or HEPES;
and/or, in step (2), the temperature used for hydration is 37-70° C.;
and/or, in step (2), the time used for hydration is 30-60 min;
and/or, in step (2), the hydrated product successively passes through polycarbonate membranes with pore sizes of 0.4 m, 0.2 m, and 0.1 m for 3-5 times.
more preferably, in step (2), the solvent used for hydration is phosphate buffer solution (PBS) at pH 7.4.

The present invention further provides a method for preparing the above liposomal drug carriers that binds to the blood cell membranes, which comprises the following steps:
1) A liposome solution is prepared, and then mixed with blood cells, followed by incubation;
2) The incubated product is centrifuged to remove the supernatant and obtain the liposomes; preferably, in step 1), the solvent for the liposome solution is PBS buffer;
and/or, in step 1), the incubation time is 1-6 h; and/or, the incubation temperature is 2-40° C.;
and/or, in step 2), the centrifugation conditions are at 3500-5000 g for 10-30 min;
more preferably, in step 1), the incubation time is 4 h; and/or, the incubation temperature is 37° C.

The present invention also provides the above liposomal drug carriers that bind to the blood cell membranes for use in the manufacture of drug carrier preparation;
preferably, the drug carrier preparation is obtained by loading medicaments into the above liposomal drug carriers that bind to the blood cell membrane;
more preferably, the medicaments are anti-tumor drugs, anti-bacterial drugs, anti-inflammatory drugs, anti-allergic drugs, and anti-viral drugs;
further preferably, the medicaments are paclitaxel, dexamethasone, and doxorubicin.

The present invention also provides a pharmaceutical preparation, which is obtained by loading a medicament into the above liposomal drug carriers that bind to the blood cell membranes;
preferably, the method for drug loading is to prepare drug-loaded liposomes by adding medicaments during the preparation of liposomes, and then according to the above method, the pharmaceutical preparation is prepared from the drug-loaded liposomes and blood cells;
more preferably, the medicaments are anti-tumor drugs, anti-bacterial drugs, anti-inflammatory drugs, anti-allergic drugs, and anti-viral drugs;
further preferably, the medicaments are paclitaxel, dexamethasone, and doxorubicin.

The present invention also provides a pharmaceutical composition, which comprises a pharmaceutical preparation mentioned above.

In the present invention, the drug carrier preparation is a novel drug preparation, which is formed after the drug carrier is loaded with drugs. The drug preparation of the present invention can be prepared into anti-tumor, anti-bacterial, anti-inflammatory, anti-allergic, and anti-viral preparations according to the type of drug loaded.

Compared with the prior art, the beneficial effects of the present invention are as follows:

The present invention prepares liposomal drug carriers that bind to the blood cell membranes, which can carry different types of medicaments, with high entrapment efficiency and drug-loading capacity, and can effectively improve drug delivery efficiency. Moreover, the drug carriers of the present invention can allow liposomes to bind to the cell membranes of bioactive blood cells, and after binding, the blood cells can still maintain their intact morphology as well as good activity and function, that can effectively prolong the half-life of a medicament, better avoid the clearance by the autoimmune system, and achieve better drug release and clinical treatment at lower doses, indicating good application prospects.

For the treatment with the traditional anti-cancer drug paclitaxel, usually, paclitaxel injection is directly added into 0.9% NaCl solution, and then administered via intravenous infusion. However, the release of histamine often leads to various adverse reactions such as facial redness, nausea, vomiting, and rash, making it difficult to effectively improve the overall quality of life for patients and resulting in poor therapeutic effects; for the subsequent paclitaxel liposome therapy, paclitaxel liposomes are used for treatment. Paclitaxel liposomes are injected into a shaker with a 5% glucose solution and shaken until fully dissolved, and then, 500 ml of 5% glucose solution is added, followed by administration by intravenous infusion, which can improve the solubility of the drug to a certain extent, prolong its pharmacological effects, and reduce the incidence of adverse reactions and the neurotoxicity of traditional paclitaxel. This is beneficial for strengthening the patient's tolerance and further improving the treatment effect. However, liposomes, as exogenous drugs, are easily attacked and cleared by the autoimmune system, resulting in low availability.

Therefore, in order to improve the circulation time of paclitaxel in the body, avoid it being killed by the immune system, and enhance its availability and therapeutic effects, the present invention has disclosed a new drug carrier and proposed a new administration way. Firstly, 20-50 ml of the patient's blood is drawn out, and RBCs are obtained by centrifugation. Then, paclitaxel liposomes are incubated with RBCs together, and the liposomes bind to the membranes of RBCs, so that the RBCs are loaded with paclitaxel. The RBCs are then infused back into the patient's body, achieving the goal of avoiding clearance by the body's immune system, prolonging the half-life, reducing then drug dosage, and obtaining the good therapeutic effect. Moreover, the ways of binding liposomes to RBCs can also be used to achieve targeted drug delivery, which can reduce the dosage of paclitaxel and the occurrence of drug resistance. By controlling the incubation time between liposomes and RBCs, two ways in which liposomes bind to the membrane of RBCs can be obtained, namely adhesion and fusion. When liposomes adhere to the surface of the RBC membrane, the RBCs deform and cause compression when passing through the capillaries, and then liposomes detach and remain in the capillaries, achieving targeted drug delivery. When liposomes fuse with the RBC membranes, drugs are released by the flow of RBCs in the blood vessels, greatly prolonging their half-lives. The membrane fusion process minimizes the adverse effects of liposomes on the cell membrane, maintains the integrity of the original cells, and better avoids the clearance of the autoimmune system, achieving the conditional control and technological ways of better drug release and clinical treatment optimization at lower doses.

Obviously, based on the above content of the present invention, according to the common technical knowledge and the conventional means in the field, other various modifications, alternations, or changes can further be made, without department from the above basic technical spirits.

With reference to the following specific examples, the above content of the present invention is further illustrated. But it should not be construed that the scope of the above subject matter of the present invention is limited to the following examples. The techniques realized based on the above content of the present invention are all within the scope of the present invention.

### DESCRIPTION OF FIGURES

FIG. 1. Detection of the surface membrane protein CD47 and apoptosis results of RBC-PTX liposomes with flow cytometry; A is surface membrane protein CD47; B is apoptosis.

FIG. 2. Growth curves of tumor volume after administration of DPPC liposomes in C57BJ/6J mice with lung transplant tumors in each group.

FIG. 3. Growth curves of tumor volume after administration of DOPE liposomes in C57BJ/6J mice with lung transplant tumors in each group.

FIG. 4. Determining the fusion results of liposomes and RBC membranes using fluorescence resonance energy transfer (FRET).

FIG. 5. Organ accumulation of RBC-PTX-liposomes (DPPC) prepared at different fusion times.

FIG. 6. Organ accumulation of RBC-PTX-liposomes (DOPE) prepared at different fusion times.

### EXAMPLES

Unless otherwise stated, the raw materials and equipment used in the examples of the present invention are known products obtained by purchasing those commercially available.

### Example 1. Preparing the Drug Carrier Preparation by Fusing Drug-Loaded Liposomes with RBC Membranes

### 1. Preparation of Paclitaxel-Loaded Liposomes

59.5 mg of dipalmitoylphosphatidylcholine (DPPC) and 3.5 mg of cholesterol (CHO) were dissolved in 3 mL of chloroform, and then 1.8 mg of paclitaxel (PTX) was added and dissolved. After that, using thin-film rehydration method, the solution was subjected to vacuum rotary evaporation at 45-50° C. for 60 min, followed by adding 5 mL of phosphate buffer solution (PBS) at pH 7.4, and hydrolyzed at 50-55° C. for 30 min. The hydrated product was successively passed through polycarbonate membranes with pore sizes of 0.4 m, 0.2 m, and 0.1 m, with 3-5 times for each membrane, to obtain PTX-loaded liposome solutions. After HPLC detection, the entrapment efficiency of PTX-loaded liposomes was as high as 91.14%±1.84%. The PTX-loaded liposome is named PTX-Liposome (DPPC).

### 2. Preparation of Dexamethasone-Loaded Liposomes

59.5 mg of dipalmitoylphosphatidylcholine (DPPC) and 3.5 mg of cholesterol (CHO) were dissolved in 3 mL of chloroform, and then 1.8 mg of dexamethasone (DEX) was added and dissolved. Subsequently, using thin-film rehydration method, the solution was subjected to vacuum rotary evaporation at 45-50° C. for 60 min, followed by adding 5 mL of phosphate buffer solution (PBS) at pH 7.4, and hydrolyzed at 50-55° C. for 30 min. The hydrated product was successively passed through polycarbonate membranes with pore sizes of 0.4 m, 0.2 am, and 0.1 am, with 3-5 times for each membrane, to obtain DEX-loaded liposome solutions. After HPLC detection, the entrapment efficiency of DEX-loaded liposomes was as high as 89.6%±1.2%. The DEX-loaded liposome is named DEX-Liposome (DPPC).

3. Fusion of Paclitaxel-Loaded Liposomes with RBC Membranes

The paclitaxel-loaded liposome solution obtained in step 1 was resuspended in PBS buffer (pH=7.4). 60 μL of paclitaxel-loaded liposome (1 μmol) solution was incubated with human RBCs (3×10⁸ RBCs) at 37° C. for 4 h. After centrifugation (3500 g, 10 min, three times), the RBCs not fused with liposomes were separated from those fused with liposomes, and the supernatant was removed to obtain liposome-fused RBC drug carrier preparation, named RBC-PTX-Liposome (DPPC). After detection, it was shown that the entrapment efficiency of PTX in paclitaxel-loaded liposomes fused with RBC membrane could reach 66.47%.

4. Fusion of Dexamethasone-Loaded Liposomes with RBC Membranes

The dexamethasone-loaded liposome solution obtained in step 2 was resuspended in PBS buffer (pH=7.4). 60 μL of dexamethasone-loaded liposome (1 μmol) solution was incubated with human RBCs (3×10⁸ RBCs) (37° C., 4 h). Then, after centrifugation (3500 g, 10 min, three times), the RBCs not fused with liposomes were separated from those fused with liposomes, and the supernatant was removed to obtain liposome-fused RBC drug carrier preparation, named RBC-DEX-Liposome (DPPC). After detection, the entrapment efficiency of DEX in dexamethasone-loaded liposomes fused with RBC membrane could reach 48.13%.

### Example 2. Preparing the Drug Carrier Preparation by Fusing Drug-Loaded Liposomes with RBC Membranes

### 1. Preparation of Paclitaxel-Loaded Liposomes

The method for preparing PTX-Liposome was the same as that of Example 1.

### 2. Preparation of Dexamethasone-Loaded Liposomes

The method for preparing DEX-Liposome was the same as that of Example 1.

3. Fusion of Paclitaxel-Loaded Liposomes with RBC Membranes

The paclitaxel-loaded liposome solution obtained in step 1 was resuspended in PBS buffer (pH=7.4). 120 μL of paclitaxel-loaded liposome (2 μmol) solution was incubated with human RBCs (3×10⁸ RBCs) at 37° C. for 4 h. After centrifugation (3500 g, 10 min, three times), the RBCs not fused with liposomes were separated from those fused with liposomes, and the supernatant was removed to obtain liposome-fused RBC drug carrier preparation, named RBC-PTX-Liposome (DPPC). After detection, it was shown that the entrapment efficiency of PTX in paclitaxel-loaded liposomes fused with RBC membrane could reach 75.20%.

4. Fusion of Dexamethasone-Loaded Liposomes with RBC Membranes

The dexamethasone-loaded liposome solution obtained in step 2 was resuspended in PBS buffer (pH=7.4). 120 μL of dexamethasone-loaded liposome (2 μmol) solution was incubated with human RBCs (3×10⁸ RBCs) (37° C., 4 h). Then, after centrifugation (3500 g, 10 min, three times), the RBCs not fused with liposomes were separated from those fused with liposomes, and the supernatant was removed to obtain liposome-fused RBC drug carrier preparation, named RBC-DEX-Liposome (DPPC). After detection, the entrapment efficiency of DEX in dexamethasone-loaded liposomes fused with RBC membrane could reach 70.82%.

### Example 3. Preparing the Drug Carrier Preparation by Fusing Drug-Loaded Liposomes with RBC Membranes

### 1. Preparation of Paclitaxel-Loaded Liposomes

The method for preparing PTX-Liposome was the same as that of Example 1.

### 2. Preparation of Dexamethasone-Loaded Liposomes

The method for preparing DEX-Liposome was the same as that of Example 1.

3. Fusion of Paclitaxel-Loaded Liposomes with RBC Membranes

The paclitaxel-loaded liposome solution obtained in step 1 was resuspended in PBS buffer (pH=7.4). 180 μL of paclitaxel-loaded liposome (3 μmol) solution was incubated with human RBCs (3×10⁸ RBCs) at 37° C. for 4 h. After centrifugation (3500 g, 10 min, three times), the RBCs not fused with liposomes were separated from those fused with liposomes, and the supernatant was removed to obtain liposome-fused RBC drug carrier preparation, named RBC-PTX-Liposome (DPPC). After detection, it was shown that the entrapment efficiency of PTX in paclitaxel-loaded liposomes fused with RBC membrane could reach 52.33%.

4. Fusion of Dexamethasone-Loaded Liposomes with RBC Membranes

The dexamethasone-loaded liposome solution obtained in step 2 was resuspended in PBS buffer (pH=7.4). 180 μL of dexamethasone-loaded liposome (3 μmol) solution was incubated with human RBCs (3×10⁸ RBCs) (37° C., 4 h). Then, after centrifugation (3500 g, 10 min, three times), the RBCs not fused with liposomes were separated from those fused with liposomes, and the supernatant was removed to obtain liposome-fused RBC drug carrier preparation, named RBC-DEX-Liposome (DPPC). After detection, the entrapment efficiency of DEX in dexamethasone-loaded liposomes fused with RBC membrane could reach 50.31%.

### Examples 4-6. Preparing the Drug Carrier Preparations by Fusing Drug-Loaded Liposomes with RBC Membranes

According to the methods described in Examples 1-3, the number of RBCs was changed to 2×10⁸, and then liposome-fused RBC drug carrier preparations RBC-PTX-Liposome (DPPC) and RBC-DEX-Liposome (DPPC) were obtained in Examples 4-6, respectively.

### Example 7. Preparing the Drug Carrier Preparation by Fusing Drug-Loaded Liposomes with RBC Membranes

### 1. Preparation of Paclitaxel-Loaded Liposomes

46.2 mg of DPPC, 13.4 mg of dioleoylphosphatidylethanolamine (DOPE), and 3.5 mg of cholesterol (CHO) were dissolved in 3 mL of chloroform, and then 1.8 mg of paclitaxel (PTX) was added and dissolved. After that, using thin-film rehydration method, the solution was subjected to vacuum rotary evaporation at 45-50° C. for 60 min, followed by adding 5 mL of phosphate buffer solution (PBS) at pH 7.4, and hydrolyzed at 50-55° C. for 30 min. The hydrated product was successively passed through polycarbonate membranes with pore sizes of 0.4 m, 0.2 m, and 0.1 m, with 3-5 times for each membrane, to obtain PTX-loaded liposome solutions comprising DOPE. The PTX-loaded liposome is named PLX-Liposome (DOPC), and abbreviated as Liposome-DOPE.

2. Fusion of Paclitaxel-Loaded Liposomes with RBC Membranes

The method of fusing PLX-loaded liposomes with RBC membranes was the same as that of Example 2, and liposome-fused RBC drug carrier preparation was obtained and named RBC-PTX-Liposome (DOPE), which was also called 4 h-RBC-lipo (DOPE).

In the following, the beneficial effects of the present invention were demonstrated by specific experimental examples.

### Experimental Example 1: The Drug Loading Capacity of Liposomes after Fusion with RBC Membranes

### 1. Experimental Methods

(1) RBC-PTX liposomes (prepared in Examples 1-6) were washed three times with deionized water, and then the supernatant was discarded after precipitation, followed by adding 900 μL of acetonitrile to damage the membrane;
(2) Centrifuging at 10000 g for 10 min;
(3) The supernatant was separated from the precipitate and detected by HPLC, to obtain the corresponding peak area and calculate the drug concentration, the entrapment efficiency, and the drug loading capacity of RBC-PTX liposomes.

### 2. Experimental Results

After calculation, the drug loading capacity and entrapment efficiency of PTX-loaded liposomes (PTX-Liposomes) fused with red blood cell (RBC) membranes are shown in Table 1.

TABLE 1
The drug loading capacity and entrapment efficiency
of PTX-Liposome fused with RBC membranes.

| | | Entrapment | |
|---|---|---|---|
| PTX-liposome | | efficiency | PTX loaded in |
| (μmol) | RBC | (EE %) | RBC (μg) |
| 1 μmol (Example 4) | 2 × 10⁸ | 51.7% | 10.44 |
| 2 μmol (Example 5) | 2 × 10⁸ | 59.20% | 23.89 |
| 3 μmol (Example 6) | 2 × 10⁸ | 40.79% | 24.70 |
| 1 μmol (Example 1) | 3 × 10⁸ | 66.47% | 13.42 |
| 2 μmol (Example 2) | 3 × 10⁸ | 75.20% | 30.36 |
| 3 μmol (Example 3) | 3 × 10⁸ | 52.33% | 31.69 |

Note:
The entrapment rate is the entrapment rate of RBC-PTX-Liposome, and PTX loaded in RBC is the amount of PTX loaded in RBC-PTX-Liposome.

According to the results in Table 1, the drug-loaded liposomes of the present invention had good entrapment efficiencies and drug loading capacities after fusion with RBC membranes.

### Experimental Example 2: Study on the Activity of RBCs after Fusion of Drug-Loaded Liposomes with RBC Membranes

### 1. Experimental Methods

### 1.1 Measurement of the Hemolysis Rates:

(1) The plasma-free hemoglobin colorimetric assay kit was ensured to be ready;
(2) The corresponding solutions were prepared according to the requirements in Table 2;

TABLE 2
Preparation of relevant solutions.

| | | Sample tube | Standard tube | Blank tube |
|---|---|---|---|---|
| Blood sample | (mL) | 0.1 | — | — |
| Hb calibrator | (mL) | — | 0.1 | — |
| Hb control | (mL) | — | — | — |
| Physiological | (mL) | — | — | 0.1 |
| saline | | | | |
| R1 | (mL) | 1 | 1 | 1 |
| R2 | (mL) | 1 | 1 | 1 |
| R3 | (mL) | 0.1 | 0.1 | 0.1 |

(3) After mixing, the reaction was carried out at 37° C. for 20 min, and then measured on a biochemical analyzer or a microplate reader with a light path of 10 mm at a wavelength of 505 nm. The blank tube was adjusted to zero, and colorimetric analysis was performed. The calculation equation:

Sample tube absorbance/Standard tube absorbance×0.1=Plasma-free hemoglobin (g/L)

### 1.2 Detection of Phosphatidylserine (PS) on the Surface of RBC Membranes:

PS is a type of phospholipid widely present in living organisms, usually located in the inner layer of cell membranes, and closely related to membrane function. During the process of cell aging and apoptosis, PS will be everted to the outer side of the cell membrane, and the eversion rate (positive expression rate) of PS is a specific indicator of cell aging. The method for detecting PS on the surface of RBC membrane was as follows:
(1) The washed RBC-PTX-liposome and pure RBCs were diluted with physiological saline to a concentration of 2×10¹⁰ cells/L;
(2) 100 μL of RBCs was transferred into a FCM tube, gently mixed with 5 μL of FITC-AnnexinV, and then incubated at room temperature in the dark for 15 min;
(3) 400 μL of isotonic PBS was added, and FCM analysis was performed within 24 h using a flow cytometer, with excitation light at 488 nm. After adding pure RBCs, RBCs were contacted and recognized using SSC and FSC, to obtain information on 10000 RBCs.

### 1.3 Detection of CD47 (Also Known as Integrin-Associated Protein) on Cell Membrane Surface:

CD47 is an important self-recognition molecule on the surface of RBC membranes. The mutual recognition and interaction between CD47 and SIRPa play a key role in maintaining RBC function and avoiding phagocytosis by macrophages. RBCs that have lost CD47 on the membrane can be recognized by the body as foreign substances and cleared, and the loss of CD47 is a hallmark event of RBC aging. The detection method for CD47 on cell membrane surface was as follows:
(1) The washed RBC-PTX-liposome and pure RBCs were diluted with physiological saline to a concentration of 2×10¹⁰ cells/L;
(2) 100 μL of RBCs, 20 μL of FITC-IgG1, and 20 μL of FITC-anti-CD47 were transferred into a FCM tube, and gently mixed;
(3) 400 μL of isotonic PBS was added, and FCM analysis was performed within 24 h using a flow cytometer, with excitation light at 488 nm. After adding pure RBCs, RBCs were contacted and recognized using SSC and FSC, to obtain information on 10000 RBCs.

### 2. Experimental Results

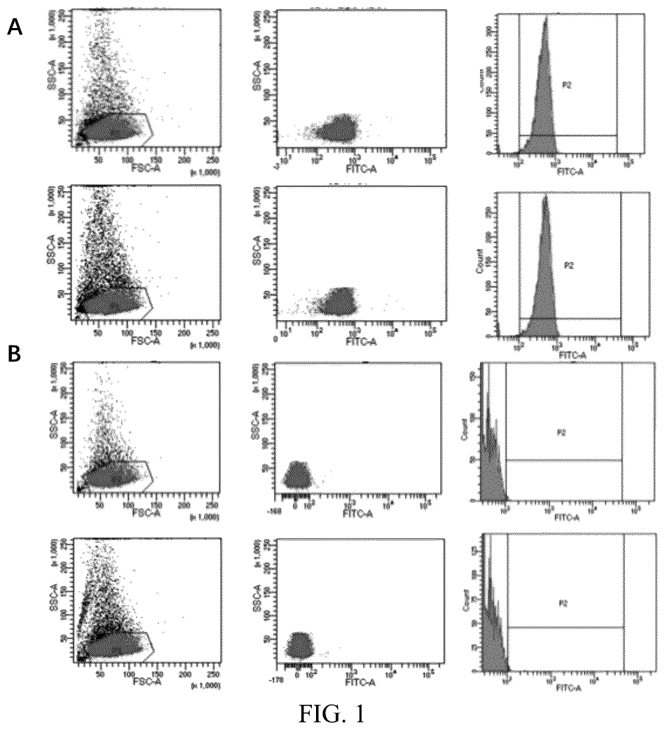

The above experiment was carried out using RBC-PTX-Liposome (RBC-lipo) prepared in Example 2 of the present invention, and the results are shown in FIGS. 1A, 1B, and Table 3. After flow cytometry analysis, it was found that in the present invention, the fusion of RBC membrane and PTX-liposome resulted in a PS positive rate of 0.22%±0.016% and a CD47 detection level of 98.37%±0.5% for the RBC membrane (The PS positive rate and CD47 detection level of the original pure RBC membrane were 0.25% and 98.74%, respectively). This indicated that in the present invention, fusing the RBC membranes with liposomes could maintain the original activities of RBCs, and even at high drug-loading levels, the morphology, cell membrane activity, or hemolysis rate of RBCs themselves were not changed. The drug carrier preparation obtained by fusing drug-loaded liposomes with RBC membranes in the present invention had RBC activity, which could better avoid the clearance of the autoimmune system and achieve better sustained release and clinical treatment effects at lower doses.

TABLE 3
The hemolysis rate (g/L) after fusion of PTX-Liposome and RBC membrane.

| | Day | | | | | | | |
|---|---|---|---|---|---|---|---|---|
| | 0 | 1 | 2 | 3 | 4 | 5 | 6 | 7 |
| RBC | 0.020 ± | 0.021 ± | 0.021 ± | 0.023 ± | 0.030 ± | 0.050 ± | 0.060 ± | 0.063 ± |
| | 0.003 | 0.002 | 0.001 | 0.003 | 0.005 | 0.006 | 0.006 | 0.003 |
| RBC-lipo | 0.021 ± | 0.0212 ± | 0.0214 ± | 0.0254 ± | 0.0308 ± | 0.0474 ± | 0.053 ± | 0.062 ± |
| | 0.003 | 0.002 | 0.005 | 0.004 | 0.004 | 0.002 | 0.002 | 0.003 |

### Experimental Example 3: The Inhibitory Effect of Drug Carrier Preparation after Fusion of Drug-Loaded Liposomes and RBC Membranes on Tumors

### 1. Experimental Methods

The lung transplant tumor models were established in C57BJ/6J mice using conventional methods, to carry out pharmacodynamic experiment in vivo, and the drug was given to mice via tail vein injection once every three days (dosage: 5 mg/kg), for a total of four doses. The changes in tumor volume and the weight of mice with transplanted tumor were monitored daily. The drug was RBC-PTX-Liposome (4 h-RBC-lipo (DPPC)) prepared in Example 2. In addition, control drugs were included. Control drug 1 was PTX-Liposome (DPPC) prepared in Example 1, while control drug 2 was the preparation (1 h-RBC-lipo(DPPC)) obtained by incubating PTX-loaded liposome solution with human HBCs at 37° C. for 1 h according to the method of Example 2.

According to the above method, Liposome-DOPE prepared in Example 7, 4 h-RBC-lipo (DOPE), and the preparation (1 h-RBC-lipo(DOPE)) obtained by incubating PTX-loaded liposome solution with human RBCs at 37° C. for 1 h according to the method of Example 7 were administered separately.

### 2. Experimental Results

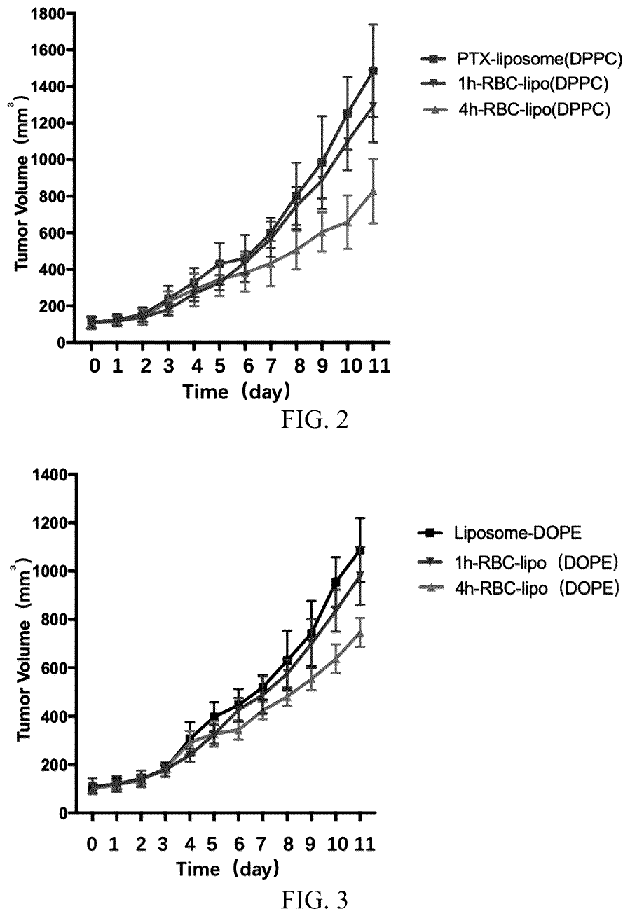

Animal experimental results indicated (FIG. 2) that compared with the PTX-Liposome group, the growth rate of tumors in the RBC-PTX-Liposome group began to slow down significantly on day 4, and the tumor volume was smaller than that of the PTX-Liposome group (P<0.05). In addition, the anti-tumor growth effect of RBC-PTX-Liposome incubated with RBCs for 4 h was significantly better than that of RBC-PTX-Liposome incubated with RBCs for 1 h.

After adding a certain proportion of dioleoylphosphatidylethanolamine DOPE to the original DPPC component of liposomes, animal experiments showed that compared with the Liposome-DOPE group, the tumor growth rate in the RBC-PTX-Liposome (DOPE) group began to significantly slow down on the fifth day, and the tumor volume was smaller than that of the Liposome-DOPE group (FIG. 3). Moreover, the therapeutic effect of 4 h-RBC-lipo (DOPE) was significantly better than that of 1 h-RBC-lipo (DOPE) (P<0.05), which was consistent with the results in FIG. 2.

The above results indicated that using RBCs as a drug delivery system could significantly improve the inhibitory effect of drug-loaded liposomes on tumors, and the effect of liposomes obtained by incubating with RBCs for 4 h was significantly better than that obtained by incubating with RBCs for 1 h.

### Experimental Example 4: The Effect of the Time for Incubating Drug-Loaded Liposomes and Red Blood Cells on Drug Carrier Preparations

### 1. Experimental Methods

1.1 Determination of Drug Loading after Incubation of PTX Liposome and RBC for Different Time
(1) PTX-liposome and washed 3×10⁸ RBCs were prepared according to the method described in Example 1;
(2) PTX-liposome solution (2 μmol) and RBCs were incubated at 37° C. for 1 h, 2 h, 3 h, and 4 h, respectively;
(3) The incubated RBC-PTX-liposome was washed three times, to which was added 1 mL of acetonitrile for membrane rupture;
(4) Centrifuging at 10000 g for 10 min;
(5) The supernatant was collected, and then subjected to HPLC analysis, to obtain the corresponding peak area, which was used to calculate the drug concentration of RBC-PTX-liposome obtained at different incubation time, as well as the entrapment efficiency and drug loading capacity.

### 1.2 Evaluation of Fusion Ability of Liposomes and RBC Membranes

Fluorescence resonance energy transfer (FRET) refers to the phenomenon of non-radioactive energy transfer from one fluorescent group to another when two fluorescent groups are sufficiently close to each other (10-100 A). The FRET degree is related to the distance between two fluorescent molecules, wherein a close distance means sufficient energy transfer; while a long distance means weak energy transfer, until energy transfer disappears. Based on the above principle, phospholipids labeled with red and green fluorescences could be used to construct liposomes together. When the distance between liposomes labeled with different fluorescent substances is close enough, FRET can be found. By measuring the changes in the emission intensity of NBD or Rh fluorescence, the degree and ability of fusion can be indirectly evaluated.

### 2. Experimental Results

By calculation, the drug loading capacity and entrapment efficiency of the drug carrier preparation obtained by incubating PTX-loaded liposomes (PTX-Liposome) with RBCs for different time are shown in Table 4.

TABLE 4
The drug loading capacity and entrapment efficiency by
incubating PTX-Liposome with RBCs for different time.

| | Incubation time | | | |
|---|---|---|---|---|
| | 1 h | 2 h | 3 h | 4 h |
| Drug loading | 10.2 ± 0.16 | 22.78 ± 1.68 | 28.44 ± 1.48 | 30.63 ± 1.34 |
| capacity | | | | |
| (μg) | | | | |
| Entrapment | 25.27 ± 0.4 | 56.44 ± 4.17 | 69.45 ± 2.83 | 75.88 ± 3.32 |
| efficiency | | | | |
| (%) | | | | |

As shown in Table 4, by changing the time of incubating liposomes and RBCs, the drug loading capacity of RBC-PTX-liposome also varied. The determination results showed that the entrapment efficiency of RBC-PTX-liposome incubated for 1 h, 2 h, 3 h, and 4 h was 25.27%±0.4, 56.44%±4.17%, 69.45%±2.83%, and 75.88%±3.32%, respectively; and the drug loading capacity was 10.2±0.16 g, 22.78±1.68 g, 28.44±1.48 g, and 30.63±1.34 g, respectively. As the incubation time extended, the entrapment efficiency and drug loading capacity also increased.

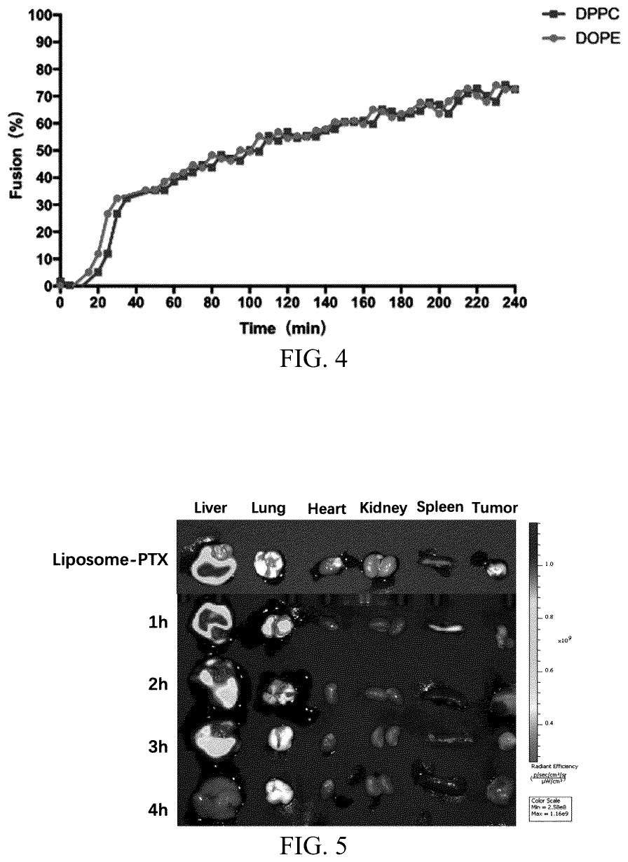

Table 5 and FIG. 4 show the results of fusing liposomes and RBC membranes, which were determined by fluorescence resonance energy transfer (FRET) method.

TABLE 5
The results of fusing liposomes and RBC membranes determined
by fluorescence resonance energy transfer (FRET) method.

| Fusion (%) | 1 h | 2 h | 3 h | 4 h |
|---|---|---|---|---|
| RBC-Lipo (DPPC) | 38.50 | 56.84 | 62.27 | 73.26 |
| RBC-Lipo (DOPE) | 40.57 | 54.52 | 63.57 | 72.61 |

The results of FRET indicated that the fusion rate (Fusion %) between liposomes and RBCs was 38.5%/40.57% after incubating for 1 h. With the prolongation of incubation time, the fusion rate gradually increased. When the incubation time was 4 h, the fusion rate reached 73.26%/72.61%, demonstrating that the effective membrane fusion between liposomes and RBC membranes was achieved under the conditions of incubating for 4 h.

### Experimental Example 5: Study on the Distribution of Drug Carrier Preparations after Fusion of Liposomes and RBC Membranes in Mice

### 1. Experimental Methods

(1) Drug-loaded liposome (2 μmol) solutions stained with DiI fluorescence and 3×10⁸ washed RBCs were prepared. The method for preparing drug-loaded liposomes stained with DiI fluorescence was the same as that of Example 1 or Example 7, except that 10 μL of DiI (20 mM) (cell membrane red fluorescent probe) was added for co-hydration before the hydration step, to obtain drug-loaded liposomes stained with DiI.
(2) The drug-loaded liposome solution was incubated with RBCs at 37° C. for 1 h, 2 h, 3 h, and 4 h, respectively.
(3) The incubated liposomes (drug carrier preparations) fused with RBCs were washed three times and injected into mice via tail vein at a dose of 5 kg/mg.
(4) After 6 hours, mouse organs were dissected and imaged under in vivo imaging system (IVIS), to observe organ accumulation.

### 2. Experimental Results

Based on the imaging results of living small animals (FIG. 5), under the conditions of 1 h incubation, the preparation was enriched in the liver and lungs, proving that the RBC-PTX-Liposome incubated for 1 h has the characteristic of “RBC-hitchhiking”, and liposomes bound to the RBC membrane by adhesion, and when they reached the pulmonary capillaries, they were compressed and released by external forces, thus accumulating in the lungs; the 4-hour incubation results showed that liposomes underwent membrane fusion with RBCs, making the binding between liposomes and RBCs more stable. Liposomes circulated throughout the body with RBCs but did not accumulate in any organ, greatly improving the in vivo circulation half-life and thereby enhancing therapeutic efficacy.

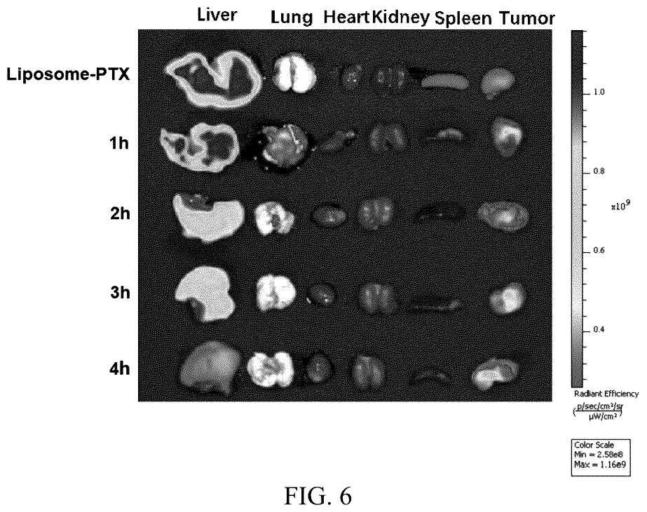

The changes in phospholipid compositions of liposomes could differently affect their organ accumulations in mice. As shown in FIG. 6, after adding a certain amount of DOPE to DPPC liposomes, compared with pure DPPC liposomes, DPPC-DOPE liposomes were incubated with RBCs for 1 hour, and then due to the pH sensitive nature of DOPE, liposomes not only enriched in the liver and lungs, but also achieved targeted delivery to tumors; after 4 hours of incubation, liposomes continued to accumulate in the liver while achieving tumor-targeting delivery. It was speculated that this result was due to the pH sensitivity of DOPE, which lead to the rupture of RBCs and then cleared by the liver.

In summary, the present invention prepared liposomal drug carriers that bound to the blood cell membranes, which could carry different types of medicaments, with high entrapment efficiency and drug-loading capacity, and could effectively improve drug delivery efficiency. Moreover, the drug carriers of the present invention could allow liposomes to bind to the cell membranes of bioactive blood cells, and after binding, the blood cells could still maintain their intact morphology as well as good activity and function, that could effectively prolong the half-life of a medicament, better avoid the clearance by the autoimmune system, and achieve better drug release and clinical treatment at lower doses, indicating good application prospects.

1-10. (canceled)

11. A liposomal drug carrier bound to the blood cell membrane, characterized in that it is obtained by mixing liposomes with blood cells and then incubating them together; the liposomes adhere to the surface of the blood cell membrane or fuse with the blood cell membrane;
the liposomes are prepared from the following raw materials at the indicated weight ratio: 50-60 parts of dipalmitoylphosphatidylcholine (DPPC) and 1-5 parts of cholesterol;
alternatively, the liposomes are prepared from the following raw materials at the indicated weight ratio: 40-50 parts of DPPC, 10-20 parts of dioleoylphosphatidylethanolamine (DOPE), and 1-5 parts of cholesterol;
the method for preparing the liposomal drug carriers bound to the blood cell membrane comprises the following steps:
1) A liposome solution is prepared, and then mixed with blood cells, followed by incubation;
2) The incubated product is centrifuged to remove the supernatant and obtain the liposomes;
in step 1), the incubation time is 4 h; the incubation temperature is 37° C.;
in step 1), the solvent for the liposome solution is PBS buffer;
in step 2), the centrifugation conditions are 3500-5000 g for 10-30 min;
the blood cells are red blood cells.

12. The liposomal drug carriers according to claim 11, characterized in that when the liposomes are incubated with blood cells, the liposome concentration is 1-6 μmol, and the number of blood cells is (1-6)×10⁸.

13. The liposomal drug carriers according to claim 12, characterized in that when the liposomes are incubated with blood cells, the liposome concentration is 2 μmol, and the number of blood cells is 3×10⁸.

14. The liposomal drug carriers according to claim 11, characterized in that the liposomes are prepared by thin-film rehydration method.

15. The liposomal drug carriers according to claim 14, characterized in that the liposomes are prepared from the following raw materials at the indicated weight ratio: 59.5 parts of DPPC and 3.5 parts of cholesterol;
alternatively, the liposomes are prepared from the following raw materials at the indicated weight ratio: 46.2 parts of DPPC, 13.4 parts of DOPE, and 3.5 parts of cholesterol.

16. The liposomal drug carriers according to claim 15, characterized in that the method for preparing the liposomes comprises the following steps:
(1) The raw materials for preparing liposomes are dissolved in an organic solvent according to the weight ratio, and then the thin film is obtained by vacuum rotary evaporation using a thin-film method;
(2) The thin film prepared in step (1) is hydrated to obtain the liposomes.

17. The liposomal drug carriers according to claim 16, characterized in that:
in step (1), the organic solvent is selected from the group consisting of methanol, trichloromethane, ethanol or isopropanol;
and/or, in step (1), the temperature for vacuum rotary evaporation used in the thin-film method is 30-70° C.;
and/or, in step (1), the time for vacuum rotary evaporation used in the thin-film method is 10-60 min;
and/or, in step (2), the solvent used for hydration is PBS or HEPES;
and/or, in step (2), the temperature used for hydration is 37-70° C.;
and/or, in step (2), the time used for hydration is 30-60 min;
and/or, in step (2), the hydrated product successively passes through polycarbonate membranes with pore sizes of 0.4 m, 0.2 m, and 0.1 m for 3-5 times.

18. The liposomal drug carriers according to claim 17, characterized in that in step (2), the solvent used for hydration is phosphate buffer solution (PBS) with pH 7.4.

19. The method for preparing the liposomal drug carriers according to claim 11 that bind to the blood cell membrane, characterized in that it comprises the following steps:
1) A liposome solution is prepared, and then mixed with blood cells, followed by incubation;
2) The incubated product is centrifuged to remove the supernatant and obtain the liposomes;
in step 1), the incubation time is 4 h; the incubation temperature is 37° C.;
and the solvent for the liposome solution is PBS buffer;
in step 2), the centrifugation conditions are 3500-5000 g for 10-30 min;

20. The liposomal drug carriers according to claim 11 that bind to the blood cell membrane for use in the manufacture of drug carrier preparation.

21. The use according to claim 20, characterized in that the drug carrier preparation is obtained by loading medicaments into the liposomal drug carriers that bind to the blood cell membrane.

22. The use according to claim 21, characterized in that the medicaments are anti-tumor drugs, anti-bacterial drugs, anti-inflammatory drugs, anti-allergic drugs, and anti-viral drugs.

23. The use according to claim 22, characterized in that the medicaments are paclitaxel, dexamethasone, and doxorubicin.

24. A pharmaceutical preparation, characterized in that it is obtained by loading a medicament into the liposomal drug carriers according to claim 11 that bind to the blood cell membrane.

25. The pharmaceutical preparation according to claim 24, characterized in that the method for drug loading is to prepare drug-loaded liposomes by adding medicaments during the preparation of liposomes, the pharmaceutical preparation is prepared from the drug-loaded liposomes and blood cells.

26. The pharmaceutical preparation according to claim 25, characterized in that the medicaments are anti-tumor drugs, anti-bacterial drugs, anti-inflammatory drugs, anti-allergic drugs, and anti-viral drugs.

27. The pharmaceutical preparation according to claim 26, characterized in that the medicaments are paclitaxel, dexamethasone, and doxorubicin.

28. A pharmaceutical composition, characterized in that it comprises a pharmaceutical preparation according to claim 24.
